(19) United States
(12) Patent Application Publication
(10) Pub. No.: US 2009/0130226 A1
(43) Pub. Date: May 21, 2009

(54) METHOD OF PRODUCING BIOMATERIALS AND BIOMATERIALS PRODUCED BY THE SAME

(76) Inventors: Yun Mo Sung, Seongnam-si (KR); Sang Ho Hwang, Uijeongbu-si (KR); Yong Won Song, Seoul (KR); Min Soek Kim, Seoul (KR)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/172,648

(22) Filed: Jul. 14, 2008

(30) Foreign Application Priority Data
Nov. 19, 2007 (KR) 1020070118139

(51) Int. Cl. A61K 33/00; H05H 1/24

(52) U.S. Cl. 424/617; 427/569

(57) ABSTRACT

A biomaterial producing method and a biomaterial produced by the same are disclosed. The method includes preparing an electrolyte by dissolving monobasic potassium phosphate (KH₂PO₄) and calcium chloride (CaCl₂) in distilled water placed in an electrolytic bath, adjusting a concentration of calcium chloride (CaCl₂) in the electrolyte, immersing titanium (Ti) as an anode and stainless steel as a cathode into the electrolyte, generating plasma by applying current and voltage to the anode and the cathode to generate arc-discharge in the titanium, and coating a surface of the titanium with a hydroxyapatite (Ca₁₀(PO₄)₆(OH)₂) layer through micro-arc oxidation using the plasma. The hydroxyapatite layer can be thickly formed at low cost and has improved crystallinity.

### CROSS-REFERENCE TO RELATED APPLICATION

The present application claims priority to Korean Patent Application No. 10-2007-118139 filed on Nov. 19, 2007, the entire disclosure of which is incorporated herein by reference.

### BACKGROUND OF THE INVENTION

1. Field of the Invention

The present invention relates to a method of producing biomaterials and biomaterials produced by the same, and more particularly to a method of producing biomaterials, in which a thick hydroxyapatite layer is formed at lower costs through a single process, and biomaterials produced by the same.

2. Description of the Related Art

Titanium has been widely used in dental and orthopedic implants due to its good mechanical strength and safety for the body.

However, titanium tends to form titanium dioxide (titania, TiO₂) in air, which is known to be bio-inert with respect to the body. In other words, an implant coated with titanium dioxide is not strongly and chemically combined with bone.

To solve this problem, titanium is generally coated with hydroxyapatite. Hydroxyapatite is a calcium phosphate-based material constituting the alveolar bone and exhibits good biocompatibility, so that it can reduce time taken to join the implant to the gum, thereby enhancing therapeutic effects.

The calcium phosphate-based material is separately injected into patients with weak jaw bones, thereby shortening recovery time, and has been necessarily used as a reliable auxiliary material for implant operations after recent introduction of a hydroxyapatite-coated implant to the marketplace.

With this regard, a variety of research has focused on hydroxyapatite-coated titanium.

Plasma spray coating is one commonly employed method.

In plasma spray coating, a powdered material (i.e., hydroxyapatite) to be used as a coating material is instantaneously melted upon introduction to a plasma flame at a temperature of 10,000° C. or more and is then coated onto a surface of a substrate via high velocity impaction.

Since the substrate (titanium) has a lower temperature, the powder fused at a high temperature is instantly coated onto the substrate surface.

In plasma spray coating, however, since molten particles are deposited on metal, it is difficult to control ingredients and crystallinity of hydroxyapatite and the coated material on the metal substrate forms an amorphous and unstable phase composed of tetracalcium phosphate, tricalcium phosphate, etc., such that it is unstable in the body. For example, it is susceptible to bio-absorption. Further, this method requires an expensive apparatus and cannot achieve uniform coating of an implant with a screw thread or other complex surface.

Sol-gel dip coating is also used in the art for this purpose.

In sol-gel dip coating, a material to be used as a coating material is prepared in a sol state and a substrate is dipped into the sol such that the coating material is attached to the substrate, followed by transforming the coating material into a gel-state thin film through hydrolysis.

The gel-state thin film can be subjected to phase transformation into a dense thin film via thermal treatment. This method has advantages in that uniform coating can be obtained even for a complicated implant structure unlike in the plasma spray coating method, and in that although this method comprises two steps, it is relatively inexpensive as compared to the plasma spray coating method.

However, the sol-gel dip coating method forms a hydroxyapatite thin film with a maximum thickness of several micrometers (μm). Although it is possible to slightly increase the thickness of the thin film through repeated dipping, not only is it difficult to achieve a thin film thickness of several tens of micrometers, but the obtained thin film obtained through repeated dipping also has a disadvantage of a weak coupling force.

Additionally, sputtering can be used. However, sputtering is disadvantageous in that it requires expensive equipment and cannot achieve uniform thick coating.

To overcome the disadvantages of the foregoing methods, a micro-arc oxidation based method has recently been used.

However, since the micro-arc oxidation primarily forms non-crystalline films exhibiting bio-instability within the body, the conventional methods require hydrothermal treatment in order to obtain crystalline hydroxyapatite. Further, even after hydrothermal treatment, very little hydroxyapatite is crystallized and most remains in an amorphous phase.

As another hydroxyapatite coating method, titanium is subjected to micro-arc oxidation to produce a coated sample, followed by dipping into a Simulated Body Fluid (SBF), thereby causing natural formation of hydroxyapatite. However, the hydroxyapatite formed by this method also has low crystallinity and unstable phases, such as tetra-calcium phosphate, tri-calcium phosphate, etc., which easily undergo bio-absorption.

### SUMMARY OF THE INVENTION

The present invention is conceived to solve the problems of the conventional techniques as described above, and an aspect of the present invention is to provide a method of producing a biomaterial, in which micro-arc oxidation is performed once without hydrothermal treatment or a Simulated Body Fluid (SBF) process, thereby improving crystallinity of a hydroxyapatite layer while simplifying the processes.

According to an aspect of the present invention, a method of producing a biomaterial 25 includes: preparing an electrolyte by dissolving monobasic potassium phosphate (KH₂PO₄) and calcium chloride (CaCl₂) in distilled water placed in an electrolytic bath; adjusting a concentration of calcium chloride (CaCl₂) in the electrolyte; immersing titanium (Ti) as an anode and stainless steel as a cathode in the electrolyte; generating plasma by applying current and voltage to the anode and the cathode to generate arc-discharge in the titanium; and coating a surface of the titanium with a hydroxyapatite (Ca₁₀(PO₄)₆(OH)₂) layer through micro-arc oxidation using the plasma.

The electrolytic bath may be made of stainless steel; the adjusting of the concentration of calcium chloride (CaCl₂) in the electrolyte may comprise adjusting a ratio of monobasic potassium phosphate (KH2PO4) to calcium chloride (CaCl2) to be in the range of 5:1 to 1:5; the voltage and the current may be applied for 1˜20 minutes; the voltage may range from 100˜900 V and the current may range from 10˜90 A; the hydroxyapatite (Ca₁₀(PO₄)₆(OH)₂) layer may be formed to have a thickness of 1˜70 μm; a calcium titanate (CaTiO₃) layer may be further interposed between the titanium and the hydroxyapatite (Ca₁₀(PO₄)₆(OH)₂) layer; the calcium titanate (CaTiO₃) layer may be formed to have a thickness of 3˜7 μm; the hydroxyapatite (Ca₁₀(PO₄)₆(OH)₂) layer may comprise hydroxyapatite particles having a particle size of 20˜100 μm; crystallinity of the hydroxyapatite (Ca₁₀(PO₄)₆(OH)₂) layer is increased by increasing the concentration of calcium chloride (CaCl₂); and the particle size of the hydroxyapatite particles constituting the hydroxyapatite (Ca₁₀(PO₄)₆(OH)₂) layer is refined and a distribution density of the hydroxyapatite particles is increased by increasing the concentration of calcium chloride (CaCl₂) ,

According to another aspect of the present invention, a biomaterial for use in humans, a material for implants, and a material for an artificial bone are produced by the method of the present invention.

### BRIEF DESCRIPTION OF THE DRAWINGS

The above and other objects, features and advantages of the present invention will become apparent from the following description of preferred embodiments given in conjunction with the accompanying drawings, in which:

FIGS. 1a to 1c are flow diagrams illustrate a method for producing a biomaterial according to one embodiment of the present invention;

FIG. 2 shows a formation mechanism of a hydroxyapatite layer in a biomaterial according to one embodiment of the present invention;

FIG. 3 shows the biomaterial according to one embodiment of the present invention;

FIG. 4 shows X-ray diffraction analysis of a biomaterial according to one embodiment of the present invention;

FIG. 5 shows crystallinity of the biomaterial according to one embodiment of the present invention;

FIG. 6 is an image showing the thickness of hydroxyapatite in a biomaterial according to one embodiment of the present invention;

FIGS. 7a to 7e are Scanning Electron Microscope (SEM) micrographs of a biomaterial according to one embodiment of the present invention;

FIGS. 8a to 8e are SEM micrographs showing particle distribution in a hydroxyapatite layer formed by the method of producing the biomaterial according to the present invention;

FIG. 9 shows results of Energy Dispersive X-ray Spectroscopy (EDS) of a biomaterial according to one embodiment of the present invention; and

FIG. 10 is a SEM micrograph showing a cross-section of the biomaterial according to one embodiment of the present invention.

### DETAILED DESCRIPTION OF THE INVENTION

Exemplary embodiments of the present invention will be described in detail with reference to the accompanying drawings hereinafter.

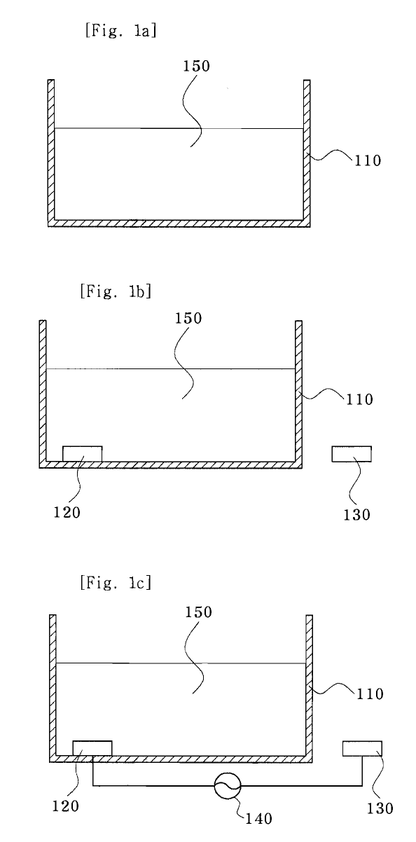

FIGS. 1a to 1c are flow diagrams illustrate a method for producing a biomaterial according to one exemplary embodiment of the present invention.

Referring to FIG. 1a, to produce a biomaterial according to the present invention, an aqueous mixture 150 of monobasic potassium phosphate (KH₂PO₄) and calcium chloride (CaCl₂) is prepared as an electrolyte having a predetermined concentration in an electrolytic bath 110.

Here, the electrolytic bath 110 may be made of metal because the electrolyte can be a weakly acidic or alkaline solution. Stainless steel is preferably used to prevent corrosion of the electrolytic bath 110.

In this embodiment, a cylindrical electrolytic bath having a diameter of 22 cm and a volume of 10 liters (L) is used. Further, the electrolytic bath 110 may be filled with different volumes of electrolytes. For example, the electrolytic bath 110 is filled with 3 L of electrolyte.

The electrolyte can be provided as the aqueous mixture 150 by dissolving powdered monobasic potassium phosphate and calcium chloride in distilled water. Here, the aqueous mixture 150 is, for example, at a temperature of 50□;

Further, the concentration of the electrolyte may be adjusted. To adjust the concentration of the electrolyte, the molarity of calcium chloride is adjusted in the range of 0.01-0.15 mol/l while monobasic potassium phosphate is maintained at a constant molarity of 0.05 (mol/l). For example, calcium chloride has a molarity of 0.05, 0.075, 0.10, 0.125 or 0.15 (mol/l).

Specifically, the concentration of the aqueous mixture 150 can be obtained by adjusting the concentrations of the electrolytes to have a ratio of calcium chloride to monobasic potassium phosphate in the range of 5:1 to 1:5.

Then, as shown in FIG. 1b, titanium (Ti) is immersed as an anode 120 and stainless steel is immersed as a cathode 130 into the aqueous mixture 150.

The anode 120 is immersed in the aqueous mixture 150 such that the entire surface of titanium to be oxidized is submerged in the aqueous mixture 150.

The cathode 130 is prepared in such a manner that a closed circuit is formed by voltage applied from the outside.

The cathode 130 may be any typical metallic material known to a person having ordinary knowledge in the art. However, the cathode 130 may employ a metallic material having a higher reduction potential than titanium used as the anode 120. Preferably, stainless steel is used as the cathode 130.

The aqueous mixture 150 is maintained at a constant temperature.

According to an exemplary embodiment, titanium (available from Hyundai Titanium Inc.) having a size of 10×40×1.0 mm is immersed as the anode 120 and stainless steel is immersed as the cathode 130 into the aqueous mixture 150.

To remove contaminants from the surface of titanium used as the anode 120, the titanium surface is rubbed with sandpaper (#1000, grit), cleaned with acetone to remove oil stains, and rinsed with distilled water before immersion into the aqueous mixture 150.

In FIG. 1c, voltage and current are applied between the anode 120 and the cathode 130, thereby crystallizing hydroxyapatite on the titanium surface.

Here, the voltage/current is applied between the anode 120 and the cathode 130 for 1˜20 minutes. Preferably, the voltage/current is applied for 5 minutes.

By applying a constant voltage between the anode 120 and the cathode 130 for 5 minutes, a voltage of 300˜350 V and a current of 30˜35 A are applied to the titanium surface.

To apply such voltage and current, a voltage of 100˜900 V and a current of 10˜90 A may be applied between the anode 120 and the cathode 130.

Accordingly, a hydroxyapatite (Ca₁₀(PO₄)₆(OH)₂) layer and a calcium titanate (CaTiO3) layer may be formed on titanium in a single process by the applied current/voltage.

Further, arc-discharge can occur in titanium when constant current and voltage are applied to the cathode and the anode for a predetermined time. The arc-discharge generates titanium plasma.

Ionic substances such as Ca, P, O, OH, etc. in the electrolyte may be formed into hydroxyapatite (Ca₁₀(PO₄)₆(OH)₂) and calcium titanate (CaTiO₃) on titanium by a single titanium plasma-based process.

As such, a hydroxyapatite layer having high crystallinity and thickness is formed on the titanium surface by the single process.

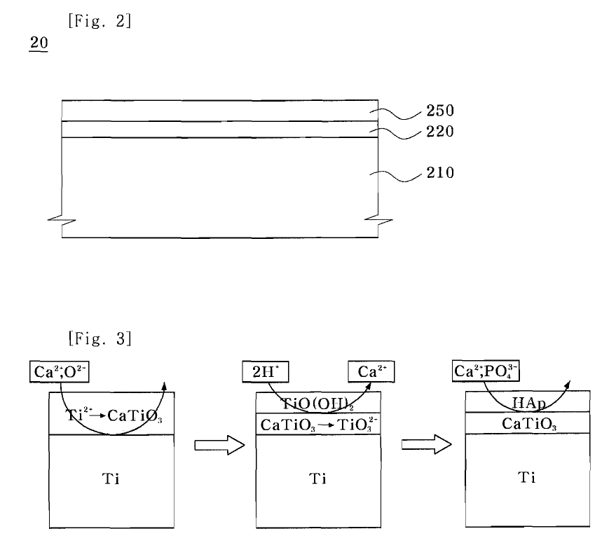

FIG. 2 shows a formation mechanism of a hydroxyapatite layer in a biomaterial according to one embodiment of the present invention and FIG. 3 shows the biomaterial according to the present invention.

Referring to FIGS. 2 and 3, the formation mechanism of the hydroxyapatite layer on a biomaterial 20 according to the embodiment of the present invention proceeds as follows:

CaCl₂ and KH₂PO₄ are dissociated into ions of Ca²⁺, Cl⁻, K⁺ and H₂PO⁴⁻ in the electrolyte. Titanium and Ca²⁺form an amorphous layer of CaTiO³ by the following reaction formulas.

Ti→Ti⁴⁺+4^{e-}

Ca²⁺+TI⁴⁺+3O²⁻→CaTiO₃

Next, H3O⁺(H⁺) ions are formed from H2PO⁴⁻ ions and are combined with CaTiO₃ ions. As a result, a TiO(OH)₂ layer is formed by ionic exchange between Ca²⁻ derived from CaTiO₃ and H3O+(H+) derived from H2PO4−.

CaTiO3+2H+→TiO(OH)2+Ca2+

The TiO(OH)₂ layer forms various Ti—OH groups, which in turn attract Ca²⁺ and PO4²⁻ from the electrolyte, finally forming crystalline hydroxyapatite (Ca₁₀(PO₄)₆(OH)₂).

The biomaterial 20 according to the present invention a CaTiO₃ coating layer 220 and a hydroxyapatite layer 250 on the surface of a titanium layer 210 provided as the anode 120.

Accordingly, the hydroxyapatite layer 250 can be thickly formed on the titanium layer 210 at low cost in a single process based on the foregoing reaction formulas.

### EXAMPLES

Hereinafter, fabrication of a biomaterial with improved crystallinity and an increased thickness using a single process according to an exemplary embodiment of the present invention will be described.

Unnecessary description of technical elements well known to those skilled in the art will be omitted.

1. X-Ray Diffraction Analysis and Crystallinity Analysis

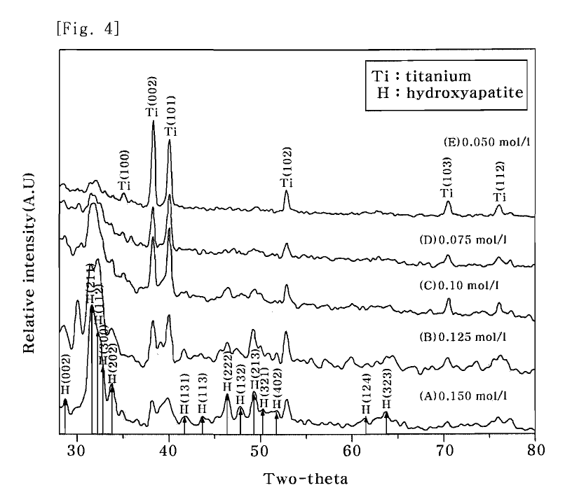

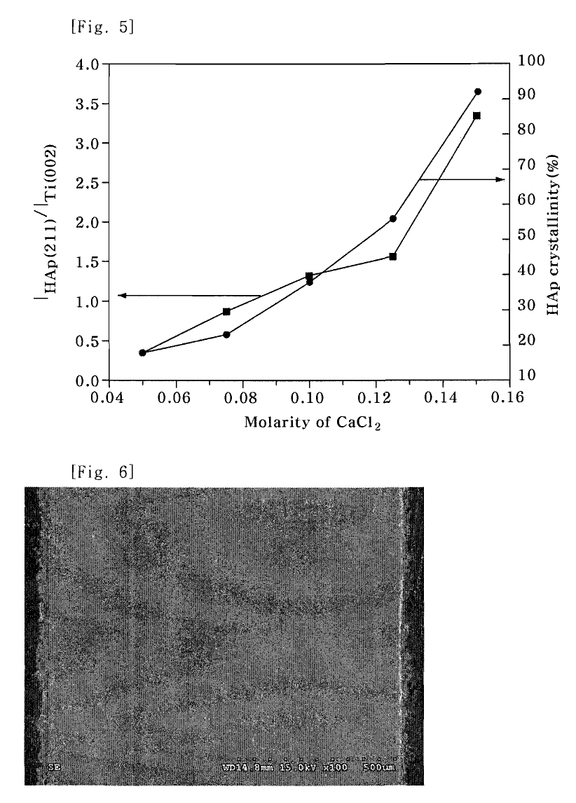

FIG. 4 shows X-ray diffraction analysis of a biomaterial according to the present invention and FIG. 5 shows crystallinity of the biomaterial according to the present invention.

For convenience, description will be made with reference to FIGS. 1a to 1c and FIGS. 2 and 3.

Structural characteristics of a biomaterial 20 depending on the concentration of calcium chloride were measured using an X-ray diffraction analyzer (XRD: Model D/MAX-2500/PC available from Rigaku, Tokyo, Japan).

In this example, X-ray diffraction analysis was performed on samples with a calcium chloride molarity in the range of 0.05-0.15 (mol/l).

From FIG. 4, it can be seen that the biomaterial 20 of the present invention exhibits more remarkable peaks of hydroxyapatite than peaks of titanium as the molarity of calcium chloride (CaCl₂) increases.

Hence, it can be understood that the peaks of hydroxyapatite become more remarkable than the peaks of titanium with an increase in molarity of calcium chloride. Specifically, the peak of titanium is higher than the peak of hydroxyapatite when calcium chloride (CaCl₂) has a molarity of 0.05 but is lower than the peak of hydroxyapatite when calcium chloride (CaCl₂) has a molarity of 0.15.

In the XRD graph, the peak of titanium is clearly shown at a molarity of 0.05, whereas the peak of hydroxyapatite is clearly shown at a molarity of 0.15.

Further, although hydroxyapatite peaks 222 and 213 do not appear at lower molarities calcium chloride (CaCl₂), theses peaks appear at higher molarities thereof.

On the other hand, for example, titanium peaks 101 and 002 are lowered as the molarity of calcium chloride (CaCl2) increase.

In FIG. 5, it can be seen that a peak height ratio of hydroxyapatite peak 211 to titanium peak 002 increases as the molarity of calcium chloride (CaCl₂) increases in the biomaterial 20 of the present invention.

A component ratio of hydroxyapatite to titanium is a height difference between hydroxyapatite peak and titanium peak in the XRD graph.

In view of a relative intensity indicated by the y-axis in the XRD graph, the peaks of hydroxyapatite exhibit lower relative intensity than the peaks of titanium at a molarity of 0.05. Thus, a peak height ratio of hydroxyapatite peak 211 to titanium peak 002 is less than 1 as shown in FIG. 5.

However, the highest peak of hydroxyapatite is higher than the highest peak of titanium at a molarity of 0.15 and a peak height ratio therebetween is 3.8. In other words, hydroxyapatite peak 211 has an intensity that can be expressed by 3.8*titanium peak 002.

Further, hydroxyapatite exhibits crystallinity that gradually increases in proportion to the molarity of calcium chloride (CaCl₂) and is about 92% when the molarity of calcium chloride (CaCl₂) reaches 0.15 (mol/l).

Thus, the crystallinity of the hydroxyapatite layer 250 becomes higher as calcium chloride (CaCl₂) increases in the biomaterial 20.

2. Microstructure

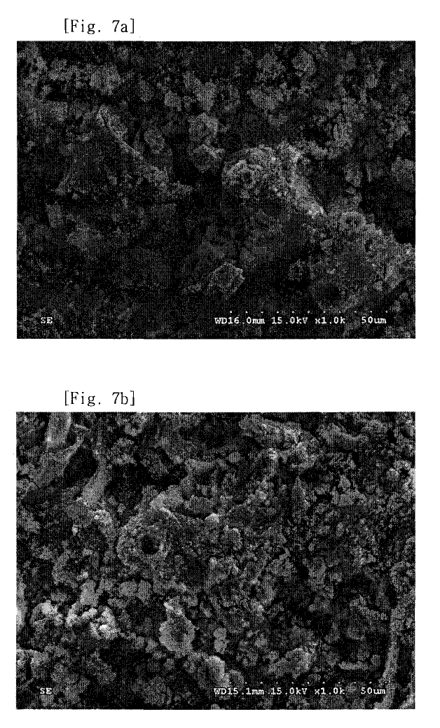

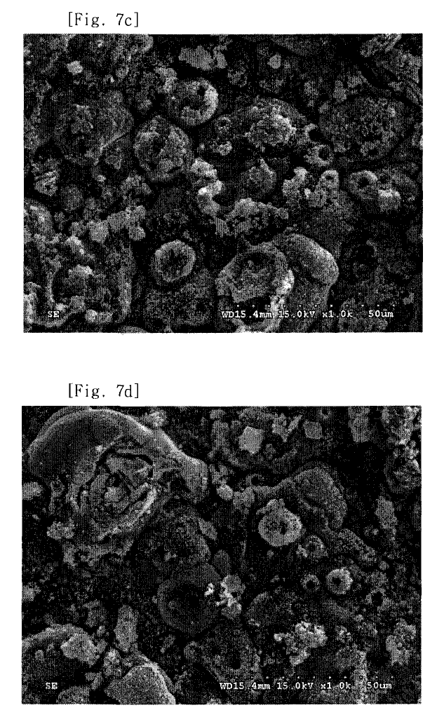

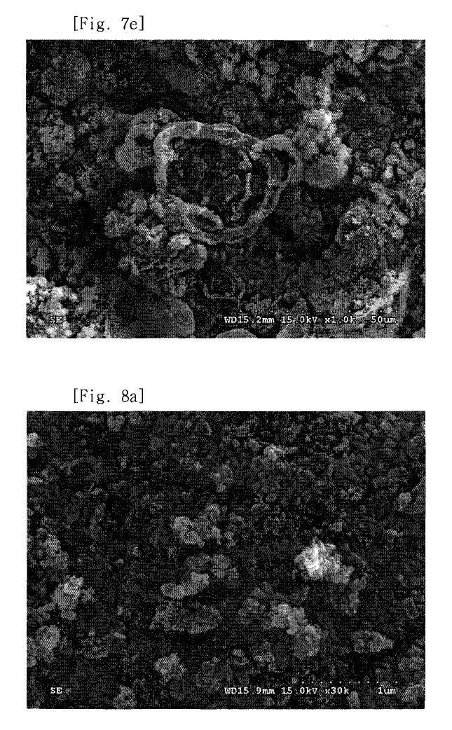

FIG. 6 is an image showing the thickness of hydroxyapatite in the biomaterial according to the present invention, and FIGS. 7a to 7e are Scanning Electron Microscope (SEM) images showing a fine structure of the biomaterial according to the present invention.

Here, Field Emission SEM (FESEM, Model S-4300 available from Hitachi, Tokyo, Japan) was used.

Referring to FIG. 6, the hydroxyapatite layer 250 is formed on the biomaterial 20 of the present invention and is produced by the method according to the present invention As a result of measuring the thickness of the hydroxyapatite layer 250, the thickness was adjustable from a thin coating layer of 1˜70 μm to a very thick coating layer.

FIG. 6 is a SEM micrograph of the hydroxyapatite layer 250 that is not polished.

Referring to FIG. 6, KH₂PO₄ has a molarity of 0.05 (mol/l), CaCl₂ has a molarity of 0.15 (mol/l), and varying molarities of CaCl₂ produce little change in thickness.

As such, in the method of the present invention, the hydroxyapatite layer 250 can be relatively thickly produced via a single process at lower costs, thereby allowing easy manufacture of biomaterial.

FIGS. 7a to 7e are micrographs of the hydroxyapatite layer 250 in the biomaterial 20 according to the present invention.

Here, these micrographs were taken while changing the molarity of CaCl₂ with respect to KH₂PO₄ having a constant molarity of 0.05.

Specifically, the micrographs of the hydroxyapatite layer 250 of the biomaterial 20 were obtained in the respective cases of CaCl₂ having a molarity of 0.05 (see FIG. 7a); CaCl₂ having a molarity of 0.075 (see FIG. 7b); CaCl₂ having a molarity of 0.1 (see FIG. 7c); CaCl₂ having a molarity of 0.125 (see FIG. 7d); and CaCl₂ having a molarity of 0.15 (see FIG. 7e).

Here, each of the micrographs shows the surface of the hydroxyapatite 250 under SEM at 1000 times magnification.

Referring to FIGS. 7a and 7b, it can be found that no specific microstructure was formed when CaCl₂ had molarities of 0.05 and 0.075. The microstructure is irregular and has a small number of pores.

Referring to FIGS. 7c and 7d, it can be found that a cellular microstructure having a size of 20˜40 μm was formed when CaCl₂ had molarities of 0.10 and 0.125. In the microstructure, the number of pores increases as compared to that in the case of the molarities of 0.05 and 0.75, and the pore has a size in the range of 2˜5 μm.

Referring to FIGS. 7d and 7e, it can be found that a rose pattern was formed when CaCl₂ had molarities of 0.125 and 0.15.

As a result, it can be understood that the pore and the microstructure of the hydroxyapatite layer 250 of the biomaterial according to the present invention are changed depending on the molarity of calcium chloride (CaCl₂).

3. Particle Distribution

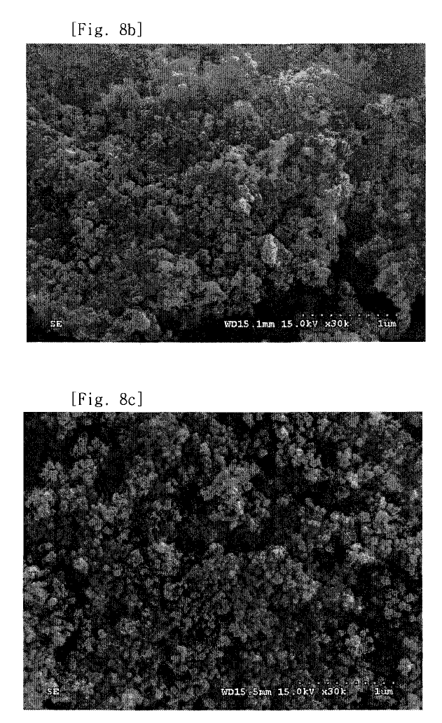

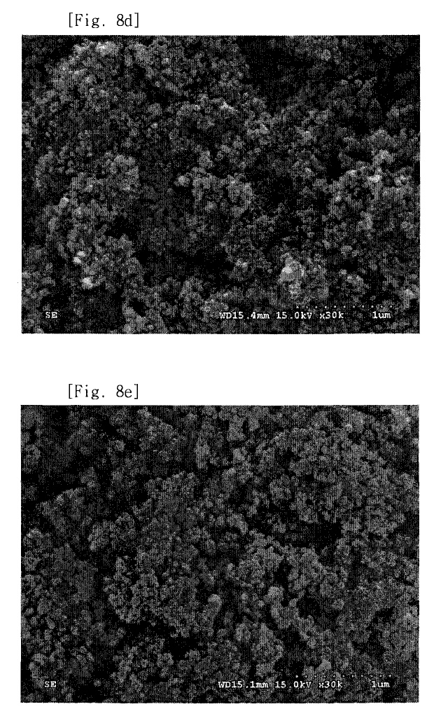

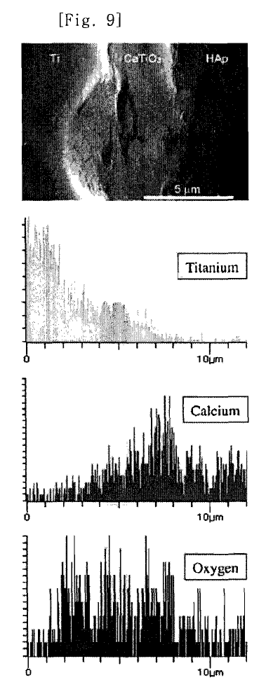

FIGS. 8a to 8e are SEM micrographs showing particle distribution in a hydroxyapatite layer formed by the method of producing a biomaterial according to the present invention.

The SEM micrographs were taken at 1000 times magnification.

The micrographs of the hydroxyapatite layer 250 of the biomaterial 20 were obtained in the respective cases of CaCl₂ having a molarity of 0.05 (see FIG. 8a); CaCl₂ having a molarity of 0.075 (see FIG. 8b); CaCl₂ having a molarity of 0.1 (see FIG. 8c); CaCl₂ having a molarity of 0.125 (see FIG. 8d); and CaCl₂ having a molarity of 0.15 (see FIG. 8e).

Referring to FIGS. 8a to 8e, it can be found that hydroxyapatite particles constituting the hydroxyapatite layer 250 coated on the biomaterial 20 of the invention were uniformly distributed on the titanium surface and had a size of 20˜100 nm.

Thus, as the molarity of calcium chloride (CaCl₂) increases, the number of nanosize particles increases, i.e., the density of particle distribution increases.

4. Energy Dispersive X-Ray Spectroscopy (EDS)

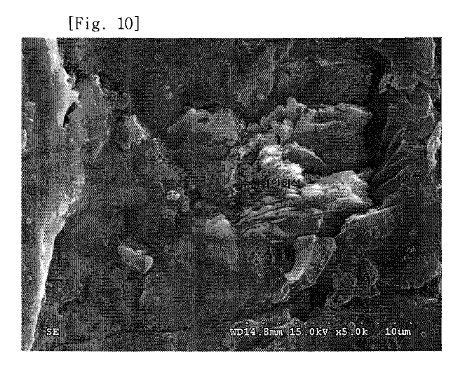

FIG. 9 shows results of Energy Dispersive X-ray Spectroscopy (EDS) of a biomaterial according to one embodiment of the present invention and FIG. 10 is an SEM micrograph showing a cross-section of a biomaterial according to one embodiment of the present invention.

Here, results of EDS are shown as a cross-section image of the biomaterial, which was obtained by SEM (EX-200 available from Hitachi, Tokyo, Japan) at 5000 times magnification. The cross-section of the biomaterial is polished.

In FIG. 9, KH₂PO₄ has a molarity of 0.05 and a CaCl₂ has a molarity of 0.10 in the biomaterial 20. Further, the cross-section was observed via energy dispersive X-ray spectroscopy (EDS) depth profiling.

The SEM micrograph and the EDS show that a CaTiO₃ layer having a thickness of 3 μm to 7 μm was formed between the titanium and hydroxyapatite layer.

Thus, the hydroxyapatite layer and the CaTiO₃ layer were sequentially formed by the method of producing a biomaterial according to the present invention, i.e., via a single process.

FIG. 10 is an SEM micrograph (X 5000) of a cross-section of a biomaterial according to one embodiment of the present invention, in which KH₂PO₄ has a molarity of 0.05 and a CaCl₂ has a molarity of 0.15 before polishing.

Here, since the cross-section of the biomaterial is too small to be sampled for the SEM, a sample of the biomaterial can be prepared by mounting the biomaterial on a substrate or the like for the purpose of SEM measurement.

An epoxy resin or the like can be prepared for easily mounting the sample on the substrate. As can be seen from the SEM image showing the crystallinity of hydroxyapatite, hydroxyapatite crystals are present near the epoxy.

According to the present invention, a biomaterial including CaTiO₃ and a hydroxyapatite layers can be produced via a single process at lower costs. Therefore, the present invention provides a biomaterial producing method and a biomaterial produced by the same method, in which the biomaterial has improved crystallinity of a hydroxyapatite layer while increasing the thickness of the hydroxyapatite layer.

Although the present invention has been described with reference to the embodiments and the accompanying drawings, the present invention is not limited to the embodiments and the drawings. It should be understood that various modifications and changes can be made by those skilled in the art without departing from the spirit and scope of the present invention as defined by the accompanying claims.

1. A method of producing a biomaterial comprising:
preparing an electrolyte by dissolving monobasic potassium phosphate (KH₂PO₄) and calcium chloride (CaCl₂) in distilled water placed in an electrolytic bath;
adjusting a concentration of calcium chloride (CaCl₂) in the electrolyte;
immersing titanium (Ti) as an anode and stainless steel as a cathode in the electrolyte;
generating plasma by applying current and voltage to the anode and the cathode to generate arc-discharge in the titanium; and
coating a surface of the titanium with a hydroxyapatite (Ca₁₀(PO₄)₆(OH)₂) layer through micro-arc oxidation using the plasma.

2. The method according to claim 1, wherein the electrolytic bath is made of stainless steel.

3. The method according to claim 1, wherein the adjusting of the concentration of calcium chloride (CaCl₂) in the electrolyte comprises adjusting a ratio of monobasic potassium phosphate (KH₂PO₄) to calcium chloride (CaCl₂) to be in the range of 5:1 to 1:5.

4. The method according to claim 1, wherein the voltage and the current are applied for 1˜20 minutes.

5. The method according to claim 1, wherein the voltage ranges from 100˜900 V and the current ranges from 10˜90 A.

6. The method according to claim 1, wherein the hydroxyapatite (Ca₁₀(PO₄)₆(OH)₂) layer is formed to have a thickness of 1˜70 μm.

7. The method according to claim 1, further comprising:
forming a calcium titanate (CaTiO₃) layer between the titanium and the hydroxyapatite (Ca₁₀(PO₄)₆(OH)₂) layer.

8. The method according to claim 7, wherein the calcium titanate (CaTiO₃) layer is formed to have a thickness of 3˜7 μm.

9. The method according to claim 1, wherein the hydroxyapatite (Ca₁₀(PO₄)₆(OH)₂) layer comprises hydroxyapatite particles having a particle size of 20˜100 nm.

10. The method according to claim 1, wherein crystallinity of the hydroxyapatite (Ca₁₀(PO₄)₆(OH)₂) layer is increased by increasing the concentration of calcium chloride (CaCl₂).

11. The method according to claim 1, wherein a particle size of hydroxyapatite particles constituting the hydroxyapatite (Ca₁₀(PO₄)₆(OH)₂) layer is refined and a distribution density of the hydroxyapatite particles is increased by increasing the concentration of calcium chloride (CaCl₂).

12. A biomaterial for use in humans produced by the method according to claim 1.

13. The biomaterial according to claim 12, wherein the biomaterial comprises:
a titanium layer;
a calcium titanate (CaTiO₃) layer on the titanium layer; and
a hydroxyapatite (Ca₁₀(PO₄)₆(OH)₂) layer on the calcium titanate (CaTiO₃) layer.

14. A material for implants produced by the method according to claim 1.

15. A material for an artificial bone produced by the method according to claim 1.
